(19) United States
(12) Patent Application Publication
(10) Pub. No.: US 2024/0345105 A1
(43) Pub. Date: Oct. 17, 2024

(54) Compositions and Methods for Detecting Analytes in Oral Fluids

(71) Applicant: George Mason Research Foundation, Inc., Fairfax, VA (US)

(72) Inventors: Lance A. Liotta, Bethesda, MD (US); Alessandra Luchini Kunkel, Burke, VA (US); Marissa Ashton Howard, Richmond, VA (US); Amanda Nicole Haymond Still, Gainesville, VA (US); Ahana Byne, Manassas, VA (US); Paul Steven Russo, Fairfax, VA (US)

(21) Appl. No.: 18/406,908

(22) Filed: Jan. 8, 2024

Related U.S. Application Data
(60) Provisional application No. 63/437,770, filed on Jan. 9, 2023.

(51) Int. Cl. G01N 33/94

(52) U.S. Cl. CPC G01N 33/94

(57) ABSTRACT

The present disclosure relates in certain aspects, to compositions and methods for stabilizing analytes of interest in an oral fluid. In another aspect, the present disclosure relates to sequestering and/or isolating analytes of interest from an oral fluid. In another aspect, the present disclosure relates to methods of detecting or quantifying the amount of an analyte of interest in an oral fluid. In another aspect, the present disclosure relates to devices and kits for the same. In certain embodiments, the analytes of interest are drugs of addiction, including tetrahydrocannabinol (THC).

### CROSS-REFERENCE TO RELATED APPLICATIONS

The present application claims priority under 35 U.S.C. § 119 (e) to U.S. Provisional Application No. 63/437,770, filed Jan. 9, 2023, which is incorporated herein by reference in its entirety.

### BACKGROUND

Oral Fluid (OF) or saliva is an ideal biofluid for testing drugs of abuse because it is noninvasive and can be collected by non-medically trained personnel. Opiates, amphetamines, cannabis, cocaine, and other prescription and non-prescription drugs are seen in higher concentrations within OF compared to blood or urine, however stability and reproducibility has been a recurring difficulty during drug testing. For example, Tetrahydrocannabinol (THC) degrades quickly at room temperatures and within plastic containers such as polystyrene. Common OF drug diagnostics include lateral flow immunoassays (LFI), ELISAs, and LC-MS. LC-MS methods are superior in determining exact concentrations, however there are large delays in diagnosis and processing protocols which reduce the recovery of analytes in a given sample.

There is thus a need in the art for compositions useful for improving the sensitivity, stability, analytical yield, and accuracy of visual immunoassays and laboratory testing methods for drug of addiction analytes, methods of use thereof, and devices and kits comprising the same. The present disclosure addresses this need.

### BRIEF SUMMARY

In one aspect, the present disclosure provides a compound of formula (I), or a salt, solvate, stereoisomer, isotopologue, or metabolite thereof, wherein R^{1a} and R^{1b} are defined elsewhere herein:

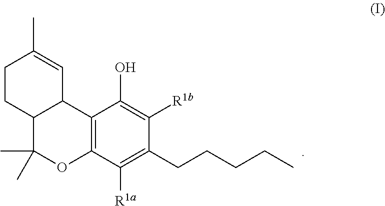

In another aspect, the present disclosure provides a composition comprising at least one drug of addiction (DOA), or a metabolite thereof, and at least one additive selected from the group consisting of an antifoaming agent, an acidic buffer, a surfactant or detergent, a mucolytic agent, an enzymatic reagent, and a mild antimicrobial agent.

In certain embodiments, the drug of addiction, or a metabolite thereof, is at least one selected from the group consisting of amphetamine, benzoylecgonine, benzodiazepines (e.g., alprazolam), cocaine, codeine, fentanyl, heroin (diacetylmorphine), hydrocodone, ketamine, methamphetamine, methadone, morphine, methylenedioxymethamphetamine (MDMA), oxycodone, phencyclidine, and tetrahydrocannabinol (THC).

In another aspect, the present disclosure provides a method for stabilizing and/or improving extractability of tetrahydrocannabinol (THC), or a metabolite thereof, in an oral fluid, the method comprising contacting an oral fluid comprising THC with at least one compound of formula (II), wherein R^{3a}, R^{3b}, R^{3c}, R^{3d}, and R^{3e} are defined elsewhere herein:

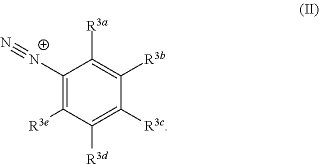

In another aspect, the present disclosure provides a method for detecting tetrahydrocannabinol (THC), or a metabolite thereof, in an oral fluid of a subject, the method comprising:
(a) contacting an oral fluid obtained from the subject with at least one compound of formula (II) to provide a stabilized sample, wherein R^{3a}, R^{3b}, R^{3c}, R^{3d}, and R^{3e} are defined elsewhere herein:

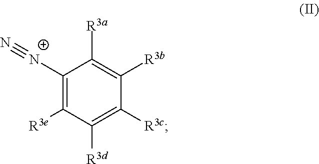

(b) applying the stabilized sample to a lateral flow assay device comprising a band comprising an immobilized THC-binding antibody; and
(c) observing coloration of the band corresponding to the immobilized THC-binding antibody.

In another aspect, the present disclosure provides a method for detecting an analyte, or a metabolite thereof, in an oral fluid of a subject, the method comprising:
(a) combining an oral fluid obtained from the subject with at least one additive selected from the group consisting of an antifoaming agent, an acidic buffer, a surfactant or detergent, a mucolytic agent, an enzymatic reagent, and a mild antimicrobial agent to provide a stabilized sample;
(b) applying the stabilized sample to a lateral flow assay device comprising at least one band comprising an immobilized antibody which specifically binds to the analyte; and
(c) observing coloration of the band corresponding to the immobilized antibody which specifically binds to the analyte.

In another aspect, the present disclosure provides a method for at least partially sequestering an analyte from an oral fluid, the method comprising:
(a) contacting the oral fluid with a porous adsorbing agent, wherein the porous adsorbing agent comprises an inert, high surface area material covalently or noncovalently associated with an affinity label; and
(b) binding the analyte to the affinity label to form an analyte bound complex.

In another aspect, the present disclosure provides a method for detecting an analyte, or a metabolite thereof, in an oral fluid of a subject, the method comprising:
(a) combining an oral fluid obtained from the subject with at least one additive selected from the group consisting of an antifoaming agent, an acidic buffer, a surfactant or detergent, a mucolytic agent, an enzymatic reagent, and a mild antimicrobial agent to provide a stabilized sample;
(b) applying the stabilized sample to a lateral flow assay device comprising at least one band comprising an immobilized antibody which specifically binds to the analyte; and
(c) observing coloration of the band corresponding to the immobilized antibody which specifically binds to the analyte.

In another aspect, the present disclosure provides a method for at least partially sequestering an analyte from an oral fluid, the method comprising:
(a) contacting the oral fluid with a porous adsorbing agent, wherein the porous adsorbing agent comprises an inert, high surface area material covalently or noncovalently associated with an affinity label; and
(b) binding the analyte to the affinity label to form an analyte bound complex.

In another aspect, the present disclosure provides an oral fluid storage device comprising a porous adsorbing agent comprising an inert, high surface area material covalently or noncovalently associated with an affinity label.

In another aspect, the present disclosure provides an oral fluid storage device comprising at least one compound of formula (II), wherein R^{3a}, R^{3b}, R^{3c}, R^{3d}, and R^{3e} are defined elsewhere herein.

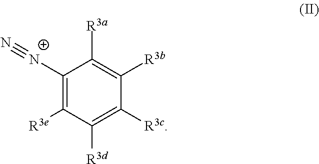

In another aspect, the present disclosure provides an oral fluid storage device comprising at least one additive selected from the group consisting of an antifoaming agent, an acidic buffer, a surfactant or detergent, a mucolytic agent, an enzymatic reagent, and a mild antimicrobial agent.

In another aspect, the present disclosure provides a kit comprising at least one device of the present disclosure and a cotton swab.

In another aspect, the present disclosure provides a kit comprising at least one device of the present disclosure and a lateral flow assay device for a drug of addiction, or a metabolite thereof.

### BRIEF DESCRIPTION OF THE FIGURES

The drawings illustrate generally, by way of example, but not by way of limitation, various embodiments of the present application.

FIG. 1 shows significant improvement in visualization of tetrahydrocannabinol (THC) in a lateral flow assay of an oral fluid sample using aryl diazonium salts. 50 ng/mL of drug of addiction analytes were spiked into an oral fluid and run through a standard commercial THC lateral flow immunoassay. The THC visualization immunoassay smeared and left a false negative without addition of an aryl diazonium salt (DT070). Adding equal amounts (50 ng/mL) of Fast Red B (DT062) and Fast Blue B (DT050) to the analyte in oral fluid produced a clearer visual immunoassay with a removed false negative signal.

FIG. 2 provides a synthetic scheme depicting reaction of THC and a generic diazonium salt. THC, comprising two electron donating oxygen substituents, undergoes an electrophilic aromatic substitution (EAS) with aryl diazonium salts at one or both of the unsubstituted positions of the aromatic ring in THC (i.e., ortho and/or para positions).

FIG. 3 depicts an exemplary liquid chromatography/multiple reaction monitoring mass spectrometry (LC/MRM MS) spectrum according to the methods described herein, wherein certain drugs of addiction (DOAs) of interest are precisely identified (i.e., morphine, amphetamine, methamphetamine, benzoylecgonine (BZE), cocaine, phencyclidine (PCP), and tetrahydrocannabinol (THC)). DOAs were processed according to the methods described herein at a concentration of 100 ng/mL.

FIGS. 4A-4G provide exemplary dose response yield curves prepared using the methods described herein with 100 ng/mL DOA with heavy isotope drug spiked in at a constant 20 ng/ml in saliva. All samples analyzed by MRM in triplicate. Each DOA dose curve had linear R values ranging from 0.939 to 1.0. The method can detect analytes of interest in low ng/ml concentrations.

FIGS. 5A-5L provide exemplary data for each drug of addiction before and after heavy isotope normalization. The data indicate significant improvements in linearity using the methods described herein.

FIGS. 6A-6E provide exemplary data indicating the reproducibility of the methods described herein for the detection of DOAs with low coefficients of variation (CV). 50 ng/ml of DOA analytes in saliva were processed according to the methods described herein. Interfering substances required by Substance Abuse and Mental Health Services (SAMSHA) were added to oral fluid samples at concentrations of 100 ng/ml and 100 ng/mL without significantly affecting the detection or measurement of DOAs in the sample according to the methods described herein.

FIGS. 7A-7B depict evaluation of the dynamic range for THC quantification within oral fluid samples using oral fluids obtained from cannabis smoking volunteers using the methods described herein.

FIG. 8 shows greater than 98% depletion/capture of CD81+100K EV bio-fluid membranous structures following incubation with an affinity matrix comprising Sudan Black B.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “adsorbing agent” as used herein refers to a solid material which is able to adsorb on its surface organic molecules dispersed or solubilized in a liquid medium, so as to sequester and eliminate such molecules by separating the adsorbing agent from the liquid medium.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “analyte” as used herein, relates to a compound, specifically to a chemical molecule, more specifically to an organic molecule. In certain embodiments, the analyte is a drug of addiction or drug of abuse.

The term “antifoaming agent” refers to a chemical molecule capable of inhibiting the formation of foam or reducing the amount of foam when mixing or shaking an aqueous solution or suspension. Antifoaming agents may comprise an organic antifoaming agent and/or a silicone-based antifoaming agent. Examples of organic antifoaming agents include Antifoam 204 and Antifoam O-30. Examples of silicone-based antifoaming agents include Antifoam A, Antifoam B, Antifoam C, Antifoam Y-30, and Sag 471. The concentration of antifoam agent is typically sufficient to ensure adequate defoaming. The concentration of an organic antifoam agent may be within the range from 0.005% to 0.01% by weight. The concentration of a silicone-based agent may be within the range from 1 ppm to 100 ppm. In certain embodiments, the anti-foaming agent comprises a carrier fluid (e.g., silicone, mineral oil, esters, and/or polyols) and a hydrophobic solid (e.g., waxes, fatty alcohols, and/or fatty acids).

The terms “antimicrobial agent” or “antimicrobial agents” refer to chemicals or other substances that kill or slow the growth of microorganisms (pathogens). Among the antimicrobial agents used today are antibacterial agents (which kill bacteria), antiviral agents (which kill viruses), antifungal agents (killing mold), and antiparasitic agents (killing parasites). The two main groups of antimicrobials are surface disinfectants, also known as “antibiotic” and “biocide”. Fungicides and antibiotics are both antibacterial agents.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl) alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2-to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The terms “drug of addiction” and “drug of abuse” are used interchangeably herein to refer to a substance that has the potential to lead to physical or psychological dependence in individuals who use it. These substances, often referred to as addictive drugs, can create a strong craving or compulsion to continue using them, even in the face of negative consequences. Non-limiting examples include, but are not limited to amphetamines (e.g., amphetamine and methamphetamine), benzodiazepines (e.g., Xanax and Valium), cocaine, hallucinogens (e.g., PCP, LSD, and psilocybin mushrooms), marijuana (e.g., tetrahydrocannabinol), opioids (e.g., heroin, oxycodone, and morphine), and sedatives (e.g., barbiturates).

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The terms “lateral flow assay” or “lateral flow assay device” as used herein, refer to any device that receives fluid, such as at least one sample, such as a bodily fluid sample, and includes at least one laterally disposed fluid transport or flow path along which various stations or sites (zones) are provided for supporting various reagents, filters and the like through which sample traverses under the influence of capillary or other applied forces and in which lateral flow assays are conducted for the detection of at least one analyte of interest. In certain embodiments, at least one site comprises an antibody which specifically binds to the analyte of interest. In certain embodiments, the site undergoes a color change or coloration upon binding of the analyte to the antibody.

The term “mucolytic agent” as used herein refers to any agent that breaks down, or hydrolyzes or liquefies mucus or mucopolysaccharides mucus sufficiently to enhance the antibacterial effect of the monobactam compounds of the composition. Suitable mucolytic agents can include, without limitation, N-acetyl-L-cysteine (MUCOSIL™; Dey Laboratories), recombinant human DNase (PULMOZYMER, Genentech, Inc.), Erdosteine (expectorant that enhances airway secretion and thus reduces viscosity of mucous), and Guaifenesin (an approved expectorant).

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “surfactant” or “surface active agent” refers to an organic chemical that when added to a liquid changes the properties of that liquid at a surface. Non-limiting examples of surfactants include but are not limited to: APG®225 Surfactant (an alkyl polyglucoside in which the alkyl group contains 8 to 10 carbon atoms and having an average degree of polymerization of 1.7); GLUCOPON®425 Surfactant (an alkyl polyglucoside in which the alkyl group contains 8 to 16 carbon atoms and having an average degree of polymerization of 1.48); GLUCOPON®625 Surfactant (an alkyl polyglucoside in which the alkyl groups contains 12 to 16 carbon atoms and having an average degree of polymerization of 1.6); APG® 325 Surfactant (an alkyl polyglucoside in which the alkyl groups contains 9 to 11 carbon atoms and having an average degree of polymerization of 1.5); GLUCOPON® 600 Surfactant (an alkyl polyglucoside in which the alkyl groups contains 12 to 16 carbon atoms and having an average degree of polymerization of 1.4); PLANTAREN® 2000 Surfactant (a C₈₋₁₆ alkyl polyglucoside in which the alkyl group contains 8 to 16 carbon atoms and having an average degree of polymerization of 1.4); and PLANTAREN® 1300 Surfactant (a C₁₂₋₁₆ alkyl polyglucoside in which the alkyl groups contains 12 to 16 carbon atoms and having an average degree of polymerization of 1.6).

### Compositions

In one aspect, the present disclosure provides a compound of formula (I), or a salt, solvate, stereoisomer, isotopologue, or metabolite thereof:

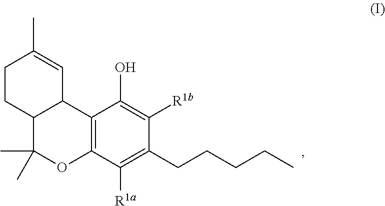

wherein:
R^{1a} and R^{1b} are each independently selected from the group consisting of H and

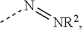

wherein at least one of R^{1a} and R^{1b} is

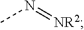

each occurrence of R² is independently selected from the group consisting of optionally substituted C₆-C₁₀ aryl and optionally substituted C₂-C₁₀ heteroaryl.

In certain embodiments, the compound of formula (I) is a compound of formula (Ia):

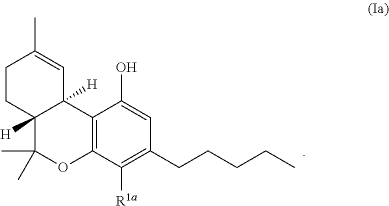

In certain embodiments, the compound of formula (I) is a compound of formula (Ib):

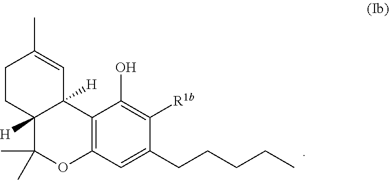

In certain embodiments, the compound of formula (I) is a compound of formula (Ic):

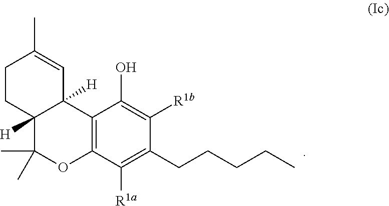

In certain embodiments, each occurrence of R² is independently:

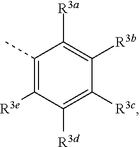

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O) (C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

In certain embodiments, R^{3a} is H. In certain embodiments, R^{3a} is CH₃. In certain embodiments, R^{3a} is OMe. In certain embodiments, R^{3a} is F. In certain embodiments, R^{3a} is Cl. In certain embodiments, R^{3a} is Br. In certain embodiments, R^{3a} is I. In certain embodiments, R^{3a} is S(═O)₂OH. In certain embodiments, R^{3a} is CN. In certain embodiments, R^{3a} is NO₂. In certain embodiments, R^{3b} is H. In certain embodiments, R^{3b} is CH₃. In certain embodiments, R^{3b} is OMe. In certain embodiments, R^{3b} is F. In certain embodiments, R^{3b} is Cl. In certain embodiments, R^{3b} is Br. In certain embodiments, R^{3b} is I. In certain embodiments, R^{3b} is S(═O)₂OH. In certain embodiments, R^{3b} is CN. In certain embodiments, R^{3b} is NO₂. In certain embodiments, R^{3c} is H. In certain embodiments, R^{3c} is CH₃. In certain embodiments, R^{3c} is OMe. In certain embodiments, R^{3c} is F. In certain embodiments, R^{3c} is Cl. In certain embodiments, R^{3c} is Br. In certain embodiments, R^{3c} is I. In certain embodiments, R^{3c} is S(═O)₂OH. In certain embodiments, R^{3c} is CN. In certain embodiments, R^{3c} is NO₂. In certain embodiments, R^{3d} is H. In certain embodiments, R^{3d} is CH₃. In certain embodiments, R^{3d} is OMe. In certain embodiments, R^{3d} is F. In certain embodiments, R^{3d} is Cl. In certain embodiments, R^{3d} is Br. In certain embodiments, R^{3d} is I. In certain embodiments, R^{3d} is S(═O)₂OH. In certain embodiments, R^{3d} is CN. In certain embodiments, R^{3d} is NO₂. In certain embodiments, R^{3e} is H. In certain embodiments, R^{3e} is CH₃. In certain embodiments, R^{3e} is OMe. In certain embodiments, R^{3e} is F. In certain embodiments, R^{3e} is Cl. In certain embodiments, R^{3e} is Br. In certain embodiments, R^{3e} is I. In certain embodiments, R^{3e} is S(═O)₂OH. In certain embodiments, R^{3e} is CN. In certain embodiments, R^{3e} is NO₂.

In another aspect, the present disclosure provides a composition comprising at least one drug of addiction (DOA), or a metabolite thereof, and at least one additive selected from the group consisting of an antifoaming agent, an acidic buffer, a surfactant or detergent, a mucolytic agent, an enzymatic reagent, and a mild antimicrobial agent.

In certain embodiments, the composition comprises at least a surfactant and an antimicrobial agent. In certain embodiments, the at least one DOA, or a metabolite thereof, is in solution or associated with a lipid containing particle. In certain embodiments, the antifoaming agent is a silicon based antifoaming agent. In certain embodiments, the surfactant or detergent comprises about 0.1% to about 5.0% of the composition. In certain embodiments, the acidic buffer comprises about 1.0% to about 5.0% of the composition. In certain embodiments, the mucolytic agent comprises about 1.0% to about 5.0% of the composition. In certain embodiments, the antimicrobial agent comprises about 0.005% to about 0.150% of the composition.

In certain embodiments, acidic buffer comprises at least one selected from the group consisting of acetic acid, ascorbic acid, and citric acid. In certain embodiments, the surfactant or detergent is a non-ionic surfactant selected from the group consisting of Triton X-100, Tween-20, sodium dodecyl sulfate (SDS), and NP-40. In certain embodiments, the surfactant or detergent is a polymeric surfactant or detergent selected from the group consisting of poloxamer 407 (P407), poloxamer (P188), poloxamer (P85), poloxamer 123 (P123), polyethylene glycol (PEG)-60, PEG-400, and PEG-1000. In certain embodiments, the mucolytic agent is selected from the group consisting of dithiothreitol and NaIO₄. In certain embodiments, the enzymatic agent is an amylase.

In certain embodiments, the drug of addiction, or a metabolite thereof, is at least one selected from the group consisting of amphetamine, benzoylecgonine, benzodiazepines (e.g., alprazolam), cocaine, codeine, fentanyl, heroin (diacetylmorphine), hydrocodone, ketamine, methamphetamine, methadone, morphine, methylenedioxymethamphetamine (MDMA), oxycodone, phencyclidine, and tetrahydrocannabinol (THC).

### Methods

In one aspect, the present disclosure provides a method for stabilizing and/or improving extractability of tetrahydrocannabinol (THC), or a metabolite thereof, in an oral fluid, the method comprising contacting an oral fluid comprising THC with at least one compound of formula (II):

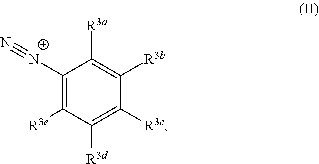

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

In certain embodiments, R^{3a} is H. In certain embodiments, R^{3a} is CH₃. In certain embodiments, R^{3a} is OMe. In certain embodiments, R^{3a} is F. In certain embodiments, R^{3a} is Cl. In certain embodiments, R^{3a} is Br. In certain embodiments, R^{3a} is I. In certain embodiments, R^{3a} is S(═O)₂OH. In certain embodiments, R^{3a} is CN. In certain embodiments, R^{3a} is NO₂. In certain embodiments, R^{3b} is H. In certain embodiments, R^{3b} is CH₃. In certain embodiments, R^{3b} is OMe. In certain embodiments, R^{3b} is F. In certain embodiments, R^{3b} is Cl. In certain embodiments, R^{3b} is Br. In certain embodiments, R^{3b} is I. In certain embodiments, R^{3b} is S(═O)₂OH. In certain embodiments, R^{3b} is CN. In certain embodiments, R^{3b} is NO₂. In certain embodiments, R^{3c} is H. In certain embodiments, R^{3c} is CH₃. In certain embodiments, R^{3c} is OMe. In certain embodiments, R^{3c} is F. In certain embodiments, R^{3c} is Cl. In certain embodiments, R^{3c} is Br. In certain embodiments, R^{3c} is I. In certain embodiments, R^{3c} is S(═O)₂OH. In certain embodiments, R^{3c} is CN. In certain embodiments, R^{3c} is NO₂. In certain embodiments, R^{3d} is H. In certain embodiments, R^{3d} is CH₃. In certain embodiments, R^{3d} is OMe. In certain embodiments, R^{3d} is F. In certain embodiments, R^{3d} is Cl. In certain embodiments, R^{3d} is Br. In certain embodiments, R^{3d} is I. In certain embodiments, R^{3d} is S(═O)₂OH. In certain embodiments, R^{3d} is CN. In certain embodiments, R^{3d} is NO₂. In certain embodiments, R^{3e} is H. In certain embodiments, R^{3e} is CH₃. In certain embodiments, R^{3e} is OMe. In certain embodiments, R^{3e} is F. In certain embodiments, R^{3e} is Cl. In certain embodiments, R^{3e} is Br. In certain embodiments, R^{3e} is I. In certain embodiments, R^{3e} is S(═O)₂OH. In certain embodiments, R^{3e} is CN. In certain embodiments, R^{3e} is NO₂.

In another aspect, the present disclosure provides a method for detecting tetrahydrocannabinol (THC), or a metabolite thereof, in an oral fluid of a subject, the method comprising:
(a) contacting an oral fluid obtained from the subject with at least one compound of formula (II) to provide a stabilized sample:

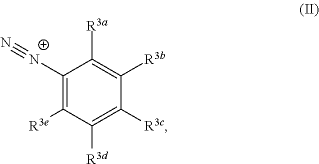

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR_{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl;
(b) applying the stabilized sample to a lateral flow assay device comprising a band comprising an immobilized THC-binding antibody; and
(c) observing coloration of the band corresponding to the immobilized THC-binding antibody.

In another aspect, the present disclosure provides a method for detecting an analyte, or a metabolite thereof, in an oral fluid of a subject, the method comprising:
(a) combining an oral fluid obtained from the subject with at least one additive selected from the group consisting of an antifoaming agent, an acidic buffer, a surfactant or detergent, a mucolytic agent, an enzymatic reagent, and a mild antimicrobial agent to provide a stabilized sample;
(b) applying the stabilized sample to a lateral flow assay device comprising at least one band comprising an immobilized antibody which specifically binds to the analyte; and
(c) observing coloration of the band corresponding to the immobilized antibody which specifically binds to the analyte.

In another aspect, the present disclosure provides a method for at least partially sequestering an analyte from an oral fluid, the method comprising:
(a) contacting the oral fluid with a porous adsorbing agent, wherein the porous adsorbing agent comprises an inert, high surface area material covalently or noncovalently associated with an affinity label; and
(b) binding the analyte to the affinity label to form an analyte bound complex.

In certain embodiments, the method further comprises washing analyte bound complex with a suitable washing solvent. In certain embodiments, the method further comprises eluting the analyte from the analyte bound complex with a suitable eluting solvent. In certain embodiments, the analyte is nonpolar, the suitable washing solvent is polar, and the suitable eluting solvent is nonpolar. In certain embodiments, the analyte is polar or ionic, and the suitable washing solvent is nonpolar, and the suitable eluting solvent is polar.

In certain embodiments, the porous adsorbing agent comprises an affinity matrix comprising a non-water imbibing, biocompatible filament. In certain embodiments, the non-water imbibing, biocompatible filament is a nylon filament.

U.S. application Ser. No. 18/354,345 filed on Jul. 18, 2023, which is incorporated herein by reference in its entirety, compositions comprising filaments functionalized with an affinity ligand.

In certain embodiments, the porous adsorbing agent comprises a hydrogel. In certain embodiments, the hydrogel is a nanoparticle.

In certain embodiments, the affinity ligand is selected from the group consisting of a dye and a lectin.

U.S. Pat. No. 10,126,304, which is incorporated herein by reference in its entirety, discloses dyes.

In certain embodiments, the dye is selected from the group consisting of an acidic dye, a basic dye, a fast dye, a metallic dye, a hydrophobic dye, and an uncharged polar dye.

In certain embodiments, the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

In certain embodiments, the dye is

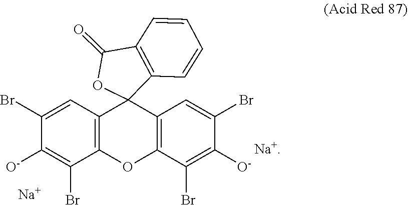

In certain embodiments, the dye is

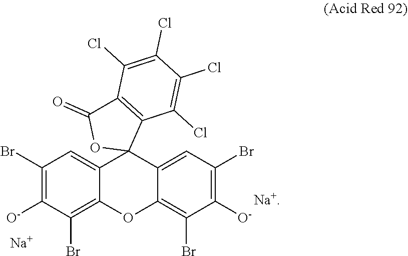

In certain embodiments, the dye is

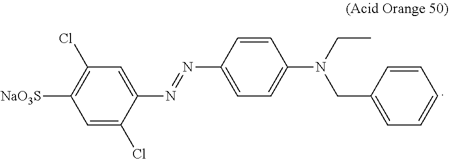

In certain embodiments, the dye is

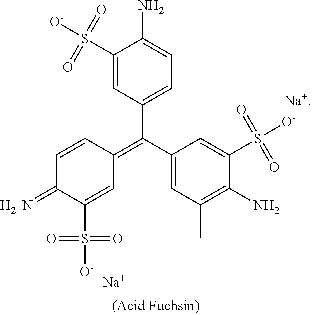

In certain embodiments, the dye is

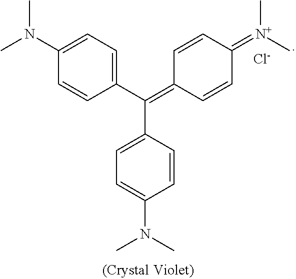

In certain embodiments, the dye is

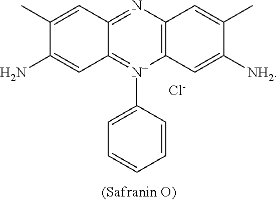 [SMILES: Cc1cc2nc3cc(C)c(N)cc3[n+](-c3ccccc3)c2cc1N ; [Cl-]]

In certain embodiments, the dye is

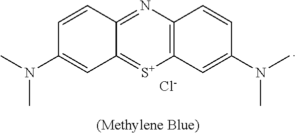

In certain embodiments, the dye is

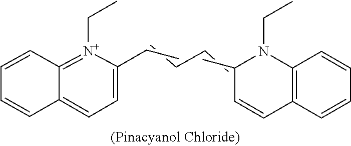

In certain embodiments, the dye is

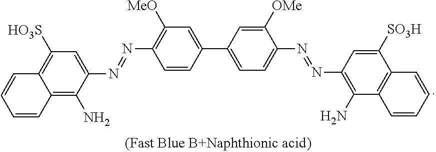 [SMILES: COc1cc(-c2ccc(/N=N/c3cc(S(=O)(=O)O)c4ccccc4c3N)c(OC)c2)ccc1/N=N/c1cc(S(=O)(=O)O)c2ccccc2c1N]

In certain embodiments, the dye is

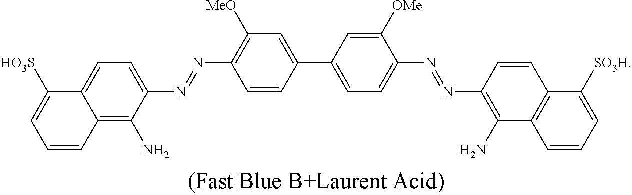 [SMILES: COc1cc(-c2ccc(/N=N/c3ccc4c(S(=O)(=O)O)cccc4c3N)c(OC)c2)ccc1/N=N/c1ccc2c(S(=O)(=O)O)cccc2c1N]

In certain embodiments, the dye is

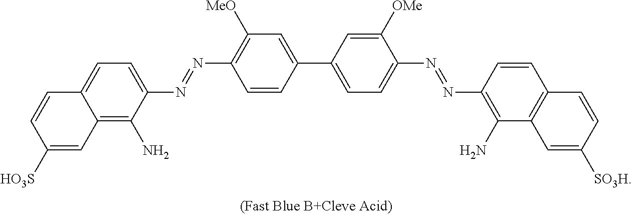 [SMILES: COc1cc(-c2ccc(/N=N/c3ccc4ccc(S(=O)(=O)O)cc4c3N)c(OC)c2)ccc1/N=N/c1ccc2ccc(S(=O)(=O)O)cc2c1N]

In certain embodiments, the dye is

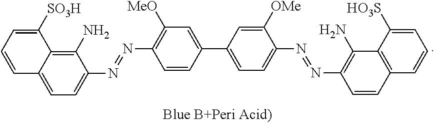 [SMILES: COc1cc(-c2ccc(/N=N/c3ccc4cccc(S(=O)(=O)O)c4c3N)c(OC)c2)ccc1/N=N/c1ccc2cccc(S(=O)(=O)O)c2c1N]

In certain embodiments, the dye is

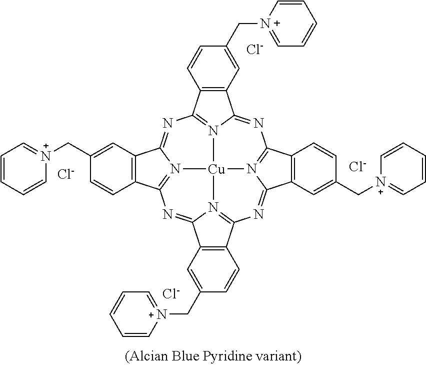

In certain embodiments, the dye is

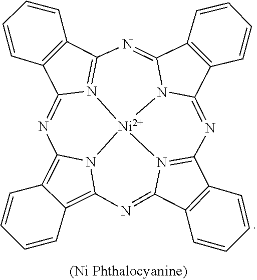

In certain embodiments, the dye is

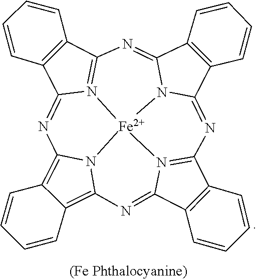

In certain embodiments, the dye is

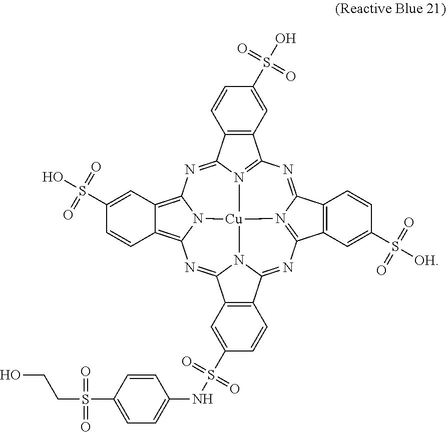

In certain embodiments, the dye is

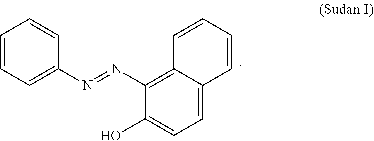 [SMILES: Oc1ccc2ccccc2c1/N=N/c1ccccc1]

In certain embodiments, the dye is

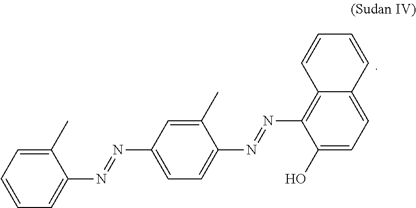 [SMILES: Cc1ccccc1/N=N/c1ccc(/N=N/c2c(O)ccc3ccccc23)c(C)c1]

In certain embodiments, the dye is

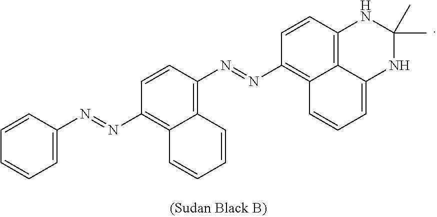 [SMILES: CC1(C)Nc2cccc3c(/N=N/c4ccc(/N=N/c5ccccc5)c5ccccc45)ccc(c23)N1]

In certain embodiments, the dye is

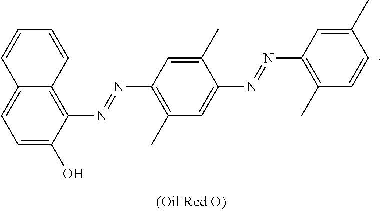 [SMILES: Cc1ccc(C)c(/N=N/c2cc(C)c(/N=N/c3c(O)ccc4ccccc34)cc2C)c1]

In certain embodiments, the dye is

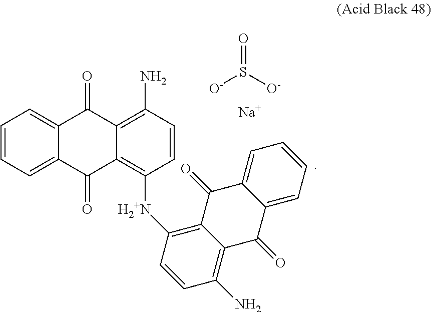

In certain embodiments, the dye is

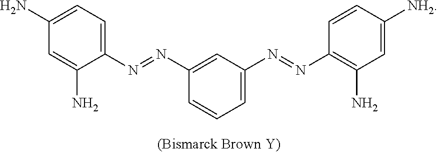 [SMILES: Nc1ccc(/N=N/c2cccc(/N=N/c3ccc(N)cc3N)c2)c(N)c1]

In certain embodiments, the dye is

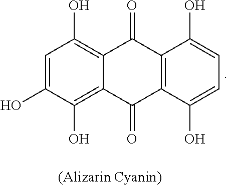 [SMILES: O=C1c2c(O)ccc(O)c2C(=O)c2c(O)c(O)cc(O)c21]

In certain embodiments, the dye is

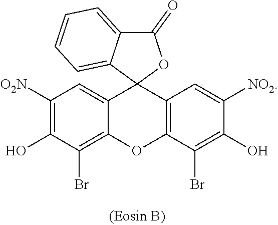 [SMILES: O=C1OC2(c3ccccc31)c1cc([N+](=O)[O-])c(O)c(Br)c1Oc1c2cc([N+](=O)[O-])c(O)c1Br]

In another aspect, the present disclosure provides a method for quantifying an amount of analyte in a sample, the method comprising:
(a) adding to the sample a known amount of at least one isotopically labeled analog of the analyte to obtain a mixture;
(b) measuring relative intensity of the analyte and relative intensity of the isotopically labeled analog of the analyte by mass spectrometry;
(c) calculating an observed relative intensity ratio of the analyte to the isotopically labeled analog of the analyte (observed L:H ratio);
(d) comparing the observed L:H ratio to a standard curve L:H ratio.

In certain embodiments, the sample is processed after addition of the at least one isotopically labeled analog of the analyte. In certain embodiments, the sample is processed before addition of the at least one isotopically labeled analog of the analyte.

In certain embodiments, the processing comprises filtration of particulate matter from the sample. In certain embodiments, the processing comprises dilution of the sample with a stabilizing buffer. In certain embodiments, the processing comprises dilution of the sample with a pH buffer. In certain embodiments, the processing comprises silica gel chromatography of the sample.

In certain embodiments, the stabilization buffer comprises a surfactant or detergent. In certain embodiments, the stabilization buffer comprises at least one selected from the group consisting of 3-((3-cholamidopropyl) dimethylammonio)-1-propanesulfonate (CHAPS), RapiGest, Tween-20, and NP-40.

In certain embodiments, the pH buffer comprises ammonium hydroxide or formic acid.

In certain embodiments, the sample is loaded onto a silica gel column and eluted with a solvent. In certain embodiments, the solvent is selected from the group consisting of hexanes, ethyl acetate, methyl tert-butyl ether (MTBE), dichloromethane, and iso-propyl alcohol, or a mixture thereof.

In certain embodiments, the eluted sample is dried under inert gas. In certain embodiments, the inert gas is N₂ (g). In certain embodiments, the sample is dried at a temperature of about 40° C.

### Devices

In one aspect, the present disclosure provides an oral fluid storage device comprising a porous adsorbing agent comprising an inert, high surface area material covalently or noncovalently associated with an affinity label.

In certain embodiments, the porous adsorbing agent comprises an affinity matrix comprising a non-water imbibing, biocompatible filament. In certain embodiments, the non-water imbibing, biocompatible filament is a nylon filament.

In certain embodiments, the porous adsorbing agent comprises a hydrogel. In certain embodiments, the hydrogel is a nanoparticle.

In certain embodiments, the affinity ligand is selected from the group consisting of a dye and a lectin.

In certain embodiments, the dye is selected from the group consisting of an acidic dye, a basic dye, a fast dye, a metallic dye, a hydrophobic dye, and an uncharged polar dye.

In certain embodiments, the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

In certain embodiments, the device is a sealable vessel.

In certain embodiments, the porous adsorbing agent is contained in the sealable vessel.

In certain embodiments, at least one compound of formula (II):

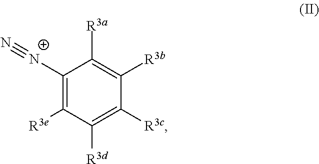

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R_{B}), OC(═O)R_{A}, N(R^{A})C(═O)R^{B}, S(═O)R^{A}, S(═O)₂OR^{A}, S(═O)₂N(R^{A})(R_{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

In certain embodiments, the device comprises a sealable inlet, optionally wherein the device further comprises a sealable outlet.

In certain embodiments, the device comprises an optionally removable filter positioned at the sealable inlet and/or at the sealable outlet.

In another aspect, the present disclosure provides an oral fluid storage device comprising at least one compound of formula (II):

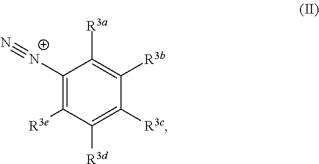

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O) (C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

In certain embodiments, the device is a sealable vessel.

In another aspect, the present disclosure provides an oral fluid storage device comprising at least one additive selected from the group consisting of an antifoaming agent, an acidic buffer, a surfactant or detergent, a mucolytic agent, an enzymatic reagent, and a mild antimicrobial agent.

In certain embodiments, the device is a sealable vessel.

### Kits

In one aspect, the present disclosure provides a kit comprising at least one device of the present disclosure and a cotton swab.

In another aspect, the present disclosure provides a kit comprising at least one device of the present disclosure and a lateral flow assay device for a drug of addiction, or a metabolite thereof.

In certain embodiments, the kit further comprises a cotton swab.

In certain embodiments, the drug of addiction, or a metabolite thereof, is selected from the group consisting of amphetamine, benzoylecgonine, benzodiazepines (e.g., alprazolam), cocaine, codeine, fentanyl, heroin (diacetylmorphine), hydrocodone, ketamine, methamphetamine, methadone, morphine, methylenedioxymethamphetamine (MDMA), oxycodone, phencyclidine, and tetrahydrocannabinol (THC).

### Examples

Various embodiments of the present application can be better understood by reference to the following Examples which are offered by way of illustration. The scope of the present application is not limited to the Examples given herein.

### Example 1: Improved Stabilization and/or Extractability of Drug Analytes from Oral Fluids (OFs)

### Fast Dyes Covalently Modify THC to Reduce Degradation in OF and Improve Extraction

Tetrahydrocannabinol (THC) is a primary drug target of interest, degrades rapidly within OF when exposed to air, acid, light, higher temperatures, and binds to many polymers. THC is a lipophilic compound which is only semi-soluble in aqueous buffers. Further, THC comprises sensitive and/or labile moieties (e.g., alkene and ether) which renders the compound prone to degradation via either heat or light. The structure of THC is provided herein for convenience:

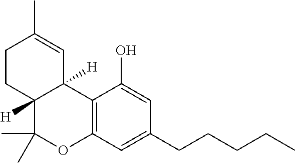 [SMILES: CCCCCc1cc(O)c2c(c1)OC(C)(C)[C@@H]1CCC(C)=C[C@@H]21]

(6aR,10aR)-6,6,9-trimethyl-3-pentyl-6a, 7,8,10a-tetrahydro-6H-benzo[c]chromen-1-ol, (THC).

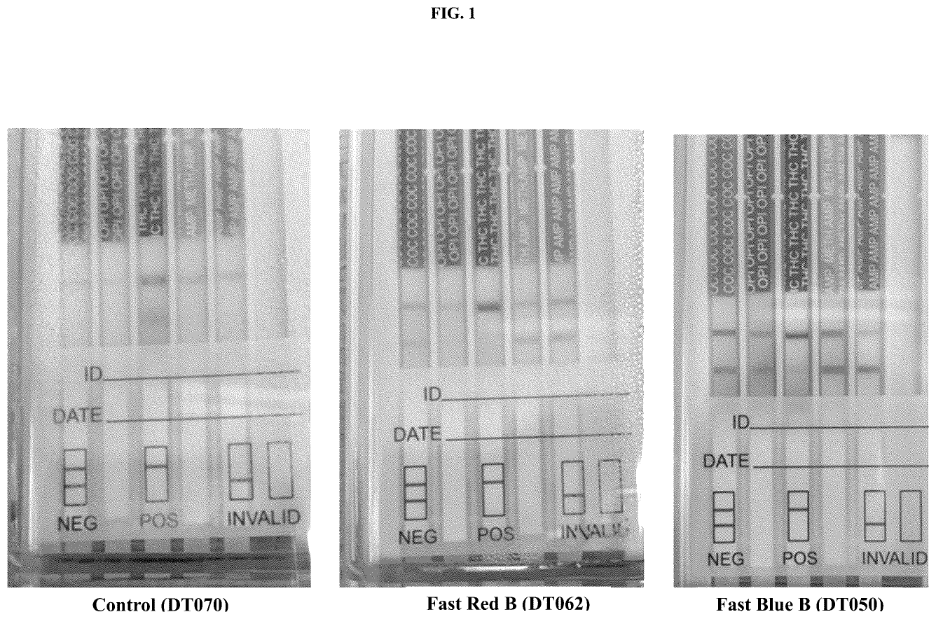

As a visual demonstration of the ability of these additives to reduce the loss of THC within OF, aryl diazonium salts (“Fast Dyes”) were combined within an OF absorbent pad or the OF collection chamber to stabilize the THC and increase its solubility in aqueous solvents. The improved solubility can reduce the amount of THC that adorbs to the plastic walls of the OF collection chamber, increasing yields of THC from OF and improving visualization in lateral flow immunoassays (FIG. 1).

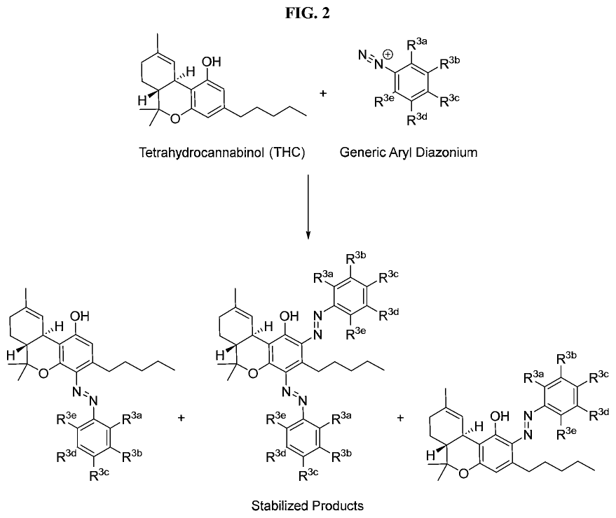

Aryl diazonium salts are known to those of ordinary skill in the art. Non-limiting, exemplary aryl diazonium salts include, but are not limited to 4-nitrobenzenediazonium, 4-nitro-2-methoxybenzenediazonium, and 4-chloro-2-methylbenzenediazonium. Key features of an aryl diazonium salt are a diazonium group and an aryl substituent. The diazonium group reacts with activated position of aryl groups in a process known as azo coupling. Azo coupling occurs between an electrophilic diazonium compound and a nucleophilic activated aryl compound. Activated aryl groups are often aryl rings with either —NH₂ or —OH substituents, which preferentially activate the ortho and para positions on the ring by reducing electron density in these positions. This allows for attack of the electrophilic diazonium compound, resulting in the formation of an azo compound product, which depending on the starting materials may be colored (FIG. 2).

The structure of THC includes an aryl ring with an activating-OH group, allowing for attack of an aryl diazonium group. When the attacking aryl diazonium salt containing very polar groups, such as —SO₃ or —NO₂, the addition of the aryl diazonium group to THC can increase its solubility in aqueous solutions such as OF and reduce the affinity of the THC for adsorption to plastics.

Formulations for Improving Drug Analyte Stability and or Extractability from OF's

OF is a complex, viscous matrix that contains mucus (e.g., polysaccharides and glycoproteins) and enzymes (e.g., amylases and lipases), that contribute to small molecule degradation. These elements are key contributors to reduced DOA analyte signal within a visual lateral flow immunoassay. A number of additives can be used to improve the visual immunoassay signal output, including but not limited to: (a) antifoaming agents; (b) acidic buffers (e.g., acetic acid, ascorbic acid, and citric acid); (c) surfactants and/or detergents (e.g., non-ionic surfactants, Triton X-100, Tween-20, SDS, and NP-40); (d) polymeric detergents (e.g., pluronic/poloxamers (407, 188, P85, and P123) and polyethylene glycol (PEG-60, PEG-400, and PEG-1000); (e) mucolytic agents (e.g., dithiothreitol and NaIO₄); (f) enzymatic reagents (e.g., amylases); and (g) mild antimicrobial agents. In certain embodiments, the formulation improves stability in an oral fluid. In certain embodiments, the formulation improves extractability from an oral fluid. In certain embodiments, the formulation improves visualization of the drug analyte in a lateral flow immunoassay.

Affinity Capture Hydrogel Nanoparticles Sequester and Protect Drugs within Oral Fluids

Affinity matrices, comprising inert, high surface area materials with an affinity label (e.g., hydrogel compositions and/or nanotraps) can be used to sequester analytes of interest by noncovalent interactions between the affinity label (e.g., dye) and the drug analyte of interest. Sequestration protects analytes within the OF from enzymatic and/or temperature related degradation. Thus, affinity matrices (e.g., nanotraps) can be used to store saliva samples for analytical tests, at which time the analyte can be eluted from the affinity matrix using an appropriate solvent or by direct swabbing. In certain embodiments, the affinity matrix may comprise a diazonium salt to further assist in stabilizing of a drug analyte of interest. Non-limiting nanotraps contemplated for use herein are described in U.S. Pat. No. 9,383,299.

Stabilization Material that Protects the DOA from Degradation Prior

An analyte extraction matrix can be incorporated in the OF collection vessel for DOA testing. This size excluding material immobilizes the OF inserted and protects the small molecule DOAs from degradation. This extraction matrix removes unwanted proteins and salts to clean up the OF for MS analysis. The incorporation of this extraction matrix within the collection vessel could allow for automation of the LC/MRM MS processing steps which would improve overall recovery and reduce potential user error.

### Example 2: Improved Precision Linearity and Yield of Drug Analyte Detection

Processing Methodology for Liquid Chromatography-Tandem Mass Spectrometry (LC-MS MS) with Multiple Reaction Monitoring (MRM)

Samples containing drugs of interest are processed and run on the Thermo Quantum Triple Quad MS (LC/MRM MS) in triplicate. Reference Stable heavy isotope analogs are spiked into known drug free OF to determine the sensitivity limit of detection. A standard curve is generated by mixing a known amount of reference heavy isotope analogs with varying amounts of the natural drug, plotting the ratio of the concentration of natural drug to that of the heavy standard isotope analog. The reference heavy isotope analogs are spiked into suspected positive OF sample. The ratio of heavy isotope analog to natural drug is calculated and fitted to the standard curve to calculate the natural drugs concentration.

Buffers can be used during the processing of the samples to reduce drug metabolization and improve limits of detection. To improve recovery of sample, the samples are diluted with a stabilization buffer/reagent which reduces analyte metabolization or loss of sample while simultaneously improving column extraction. This stabilization buffer may include, but is not limited to, a surfactant such as CHAPS (3-((3-cholamidopropyl) dimethylammonio)-1-propanesulfonate) or acid labile surfactants (Protease Max, Rapigest) or detergent like Tween-20, NP-40. Additionally, a proprietary pH adjustment buffer is added to the sample after the sample is diluted by the stabilization buffer with a specific range for each drug. The pH adjustment buffer improves recovery by prolonging the half-life of the analyte within the OF. This buffer may be, but is not limited to, ammonium hydroxide or formic acid. Finally, the extraction solution for elution of the analytes off of the column may be, but not limited to, a non-polar solvent such as hexane, ethyl acetate, MTBE, dichloromethane, isopropyl alcohol, or a mixture of the aforementioned reagents (95% dichloromethane with 5% IPA).

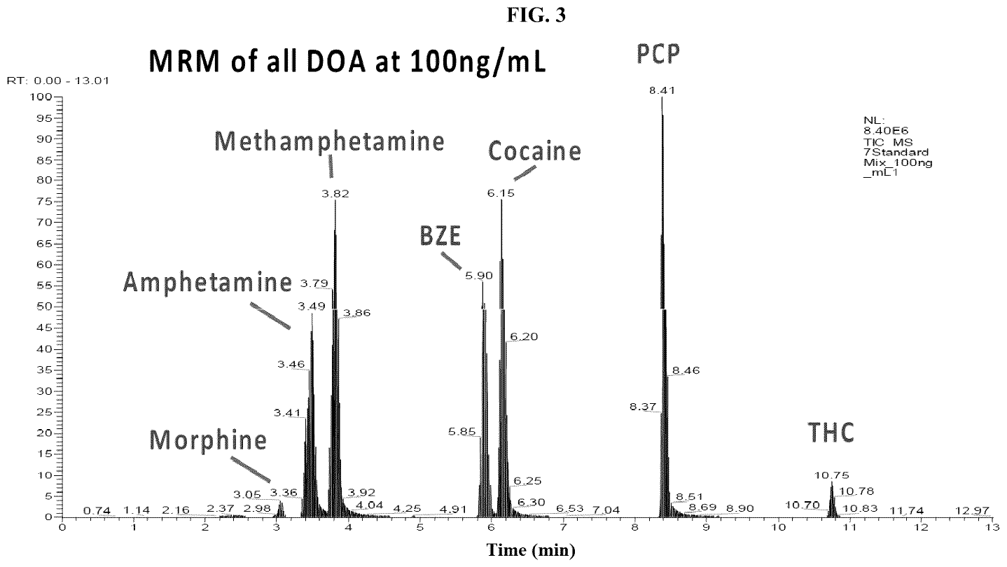

LC/MS methods described herein permit identification of key drug analytes of interest, including morphine, amphetamine, methamphetamine, benzoylecgonine (BZE), cocaine, phencyclidine (PCP), and tetrahydrocannabinol (THC) (FIG. 3).

### Exemplary Sample Processing Protocol

The present disclosure provides an exemplary oral fluid sample processing protocol. The present disclosure is not limited to the exemplary protocol described herein:
(1) Particulate matter found within the saliva is excluded.
(2) 250 μL of the sample is diluted 3:1 with stabilization buffer (750 μL buffer to 250 μL sample).
(3) A pH stabilization buffer is added to the sample.
(4) The sample is introduced to a Biotage 1 mL ISOLUTE SLE and incubated for 5 minutes at room temperature.
(5) The analyte drugs are eluted off the column twice with an extraction solution and collected.
(6) The eluted sample is dried under N₂ (g) at 40° C. for 45-60 minutes.
(7) A standard curve is generated by mixing a known amount of a reference heavy isotope analog with varying known amounts of standard of the drug of interest, and the ratio of the concentration of the natural drug to that of the heavy standard isotope is measured using a Thermo Quantum Triple Quad MS (LC/MRM MS) in triplicate.
(8) The eluted sample is reconstituted.
(9) The mixture is run on the Thermo Quantum Triple Quad MS (LC/MRM MS) in triplicate.
(10) Quantitative values of the samples are calculated based on the ratio of heavy isotope analog to analyte and fitted to the standard curve to calculate the concentration of the analyte in the sample.

In certain embodiments, a stable heavy isotope analogue is added to the sample after filtration but before addition of the stabilization buffer (i.e., between steps (1) and (2) delineated herein). In other embodiments, a stable heavy isotope analogue is added to the sample after the eluted sample is reconstituted but before the mixture is run on the Thermo Quantum Triple Quad MS (i.e., between steps (8) and (9) delineated herein).

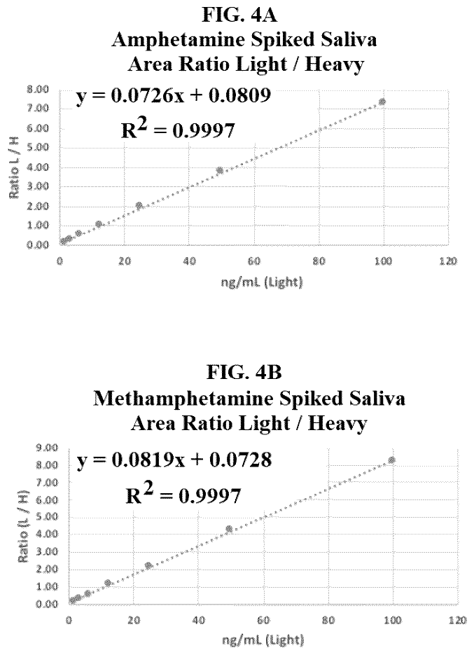

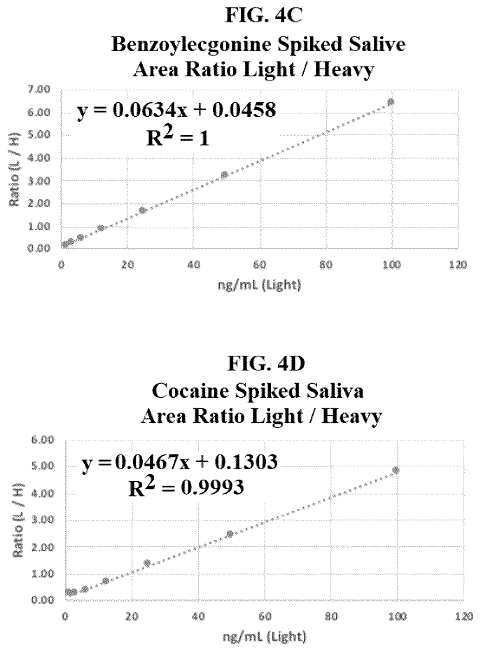

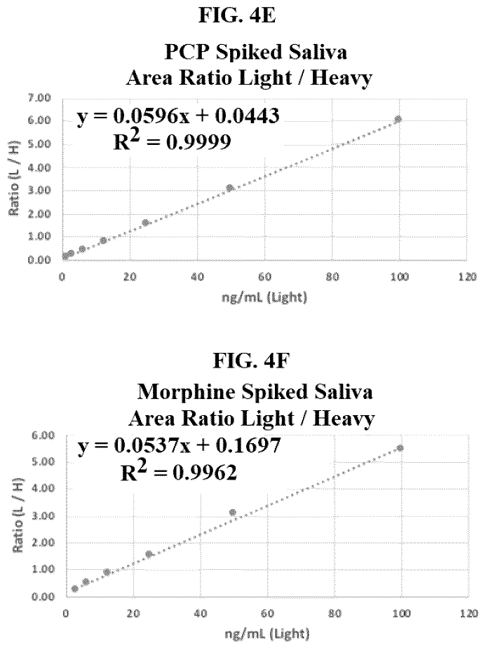

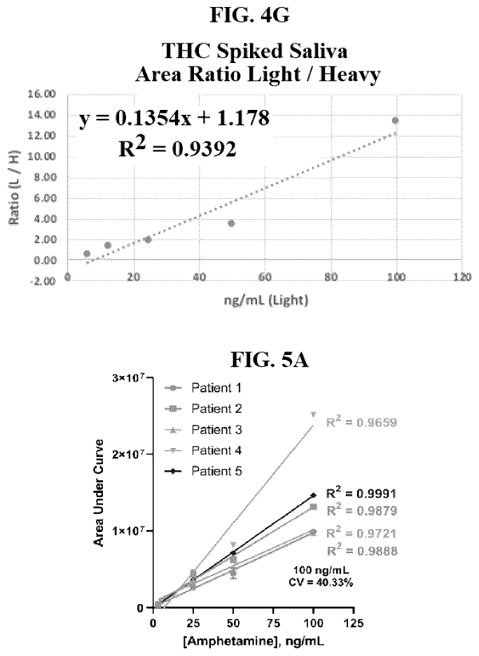

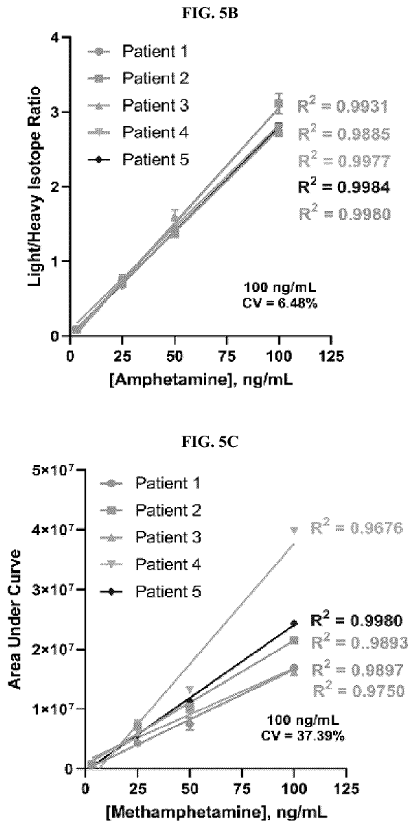

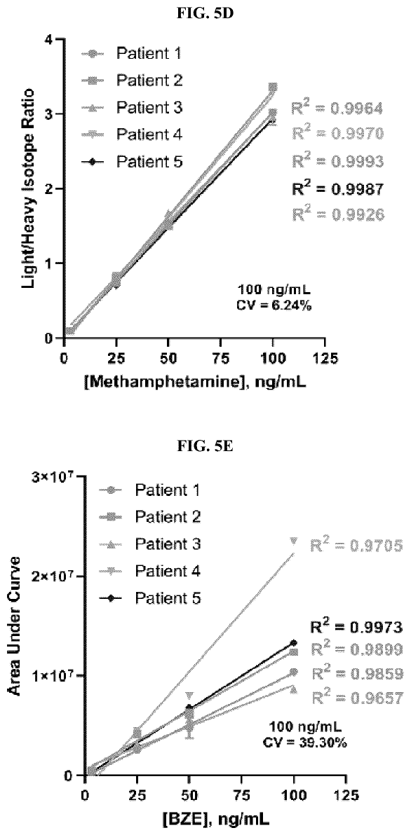

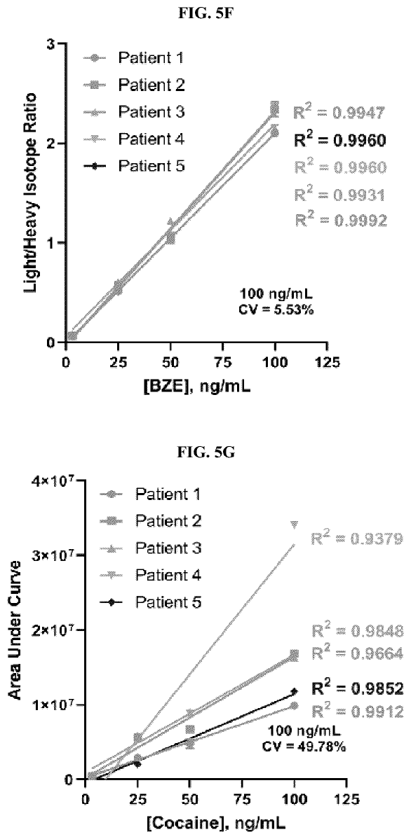

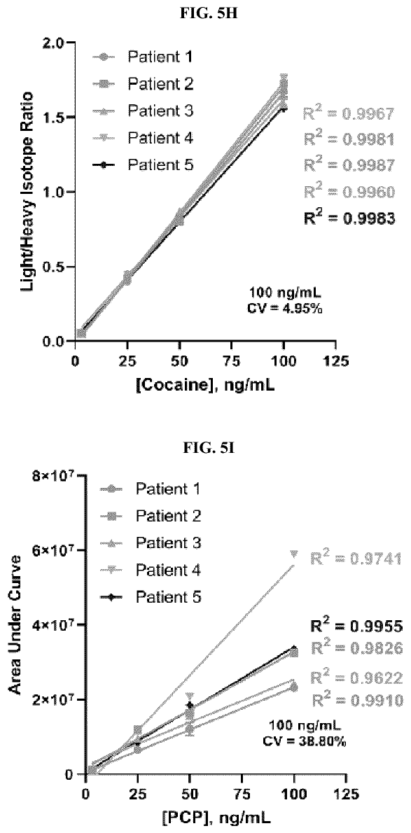

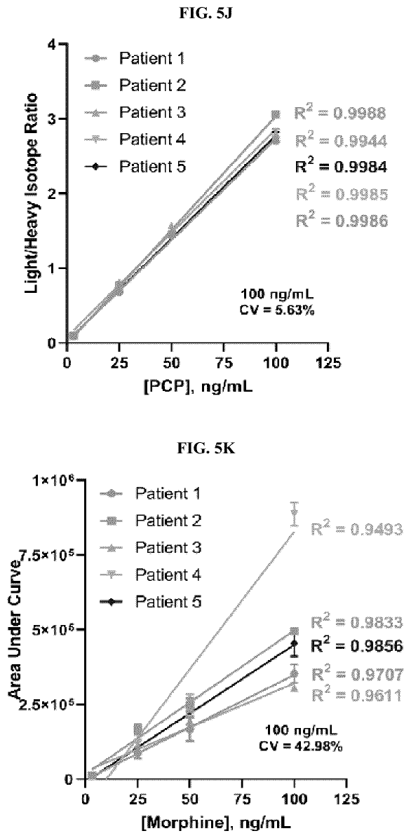

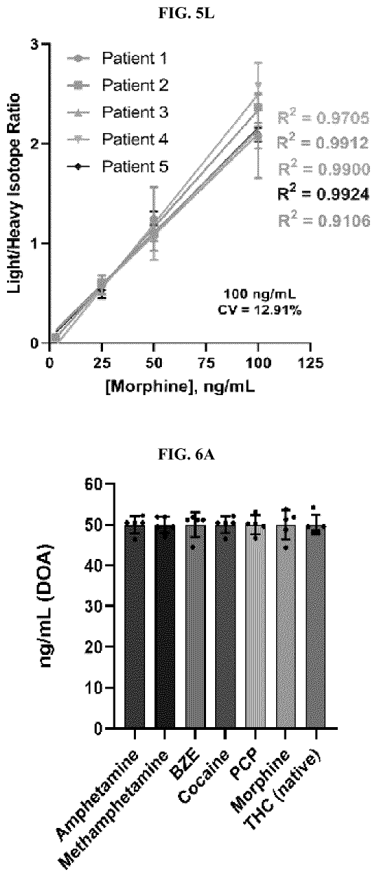

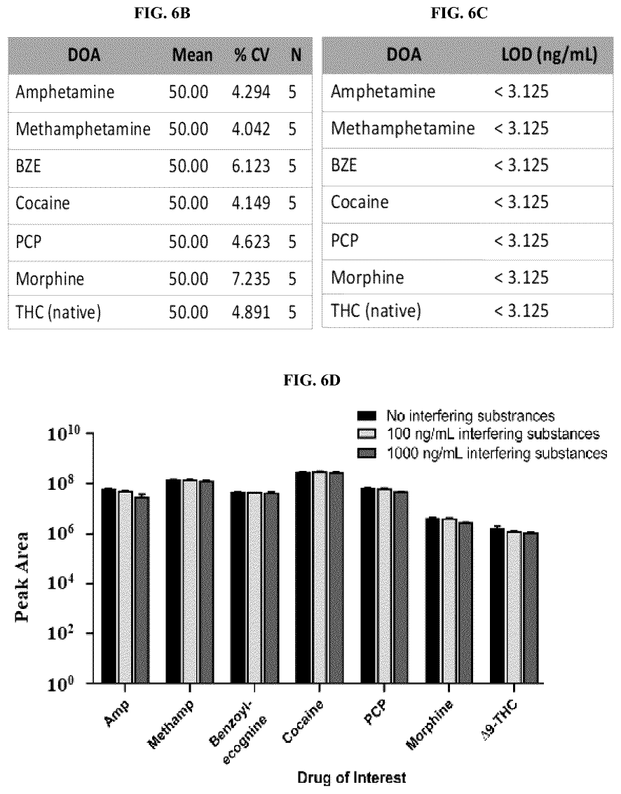

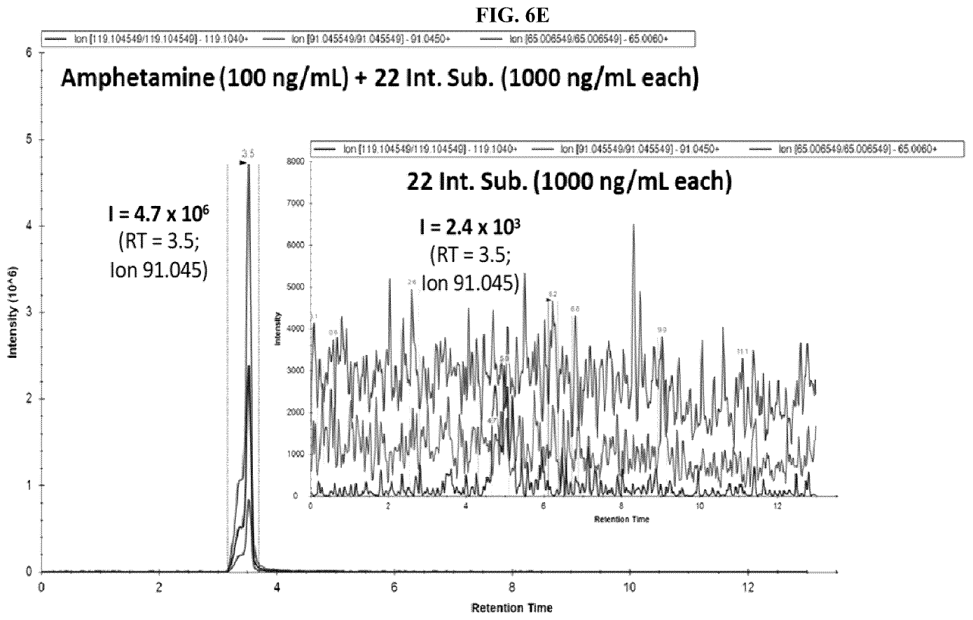

Using the heavy isotope spiking methods described herein, standard curves have been generated for amphetamine (FIG. 4A), methamphetamine (FIG. 4B), benzoylecgonine (FIG. 4C), cocaine (FIG. 4D), PCP (FIG. 4E), morphine (FIG. 4F), and THC (FIG. 4G). Further, the improved linearity of the methods described herein has been demonstrated for each of amphetamine (FIGS. 5A-5B), methamphetamine (FIGS. 5C-5D), benzoylecgonine (FIGS. 5E-5F), cocaine (FIGS. 5G-5H), PCP (FIGS. 51-5J), and morphine (FIGS. 5K-5L). Additionally, the methods described herein are highly reproducible and addition of interfering substances is not detrimental to detection of drug analytes of interest (FIGS. 6A-6E).

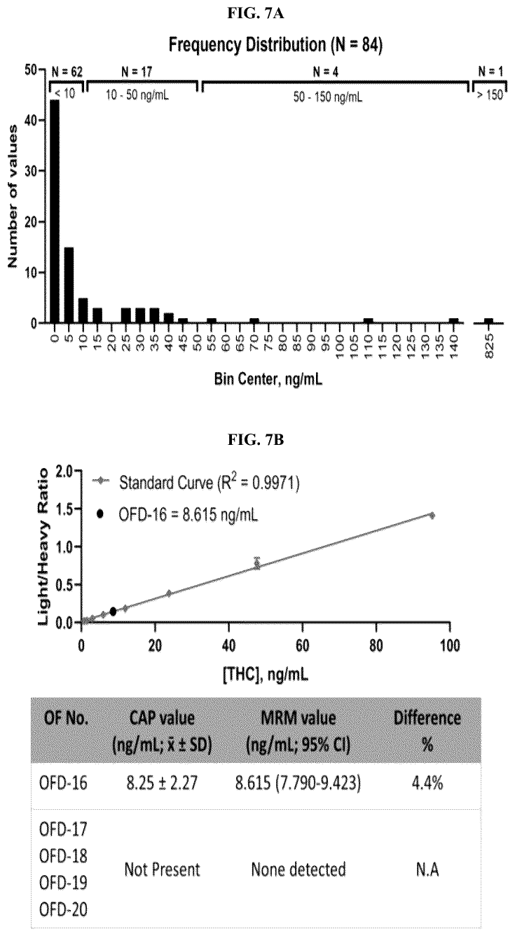

To demonstrate the utility of the method described herein, an oral fluid sample was obtained from cannabis smoking voluntary and subjected to the processing and analytical method described herein. Comparing the observed ratio of heavy THC isotope to natural THC relative intensity, as determined by LC/MRM-MS, to the generated standard curve, a concentration of 8.615 ng/ml of THC was measured in the saliva of the volunteer (FIGS. 7A-7B).

### Example 3: Isolation of Membranous and Non-Membranous Drugs of Addiction (DOAs) and Metabolites Thereof from Oral Fluid Matrices with an Affinity Matrix

Hydrophobic drugs such as THC partition into the lipid phase of membranous particles derived from the oral cavity. The same hydrophobic drugs are secreted by oral cavity cells and are incorporated into the lipid bound extracellular vesicles. Standard protocols described in the literature include centrifugation and/or filtration of these lipid associated components, which may remove the hydrophobic drug of abuse to generate a false negative. Further, the inhomogeneous nature of the hydrophobic drug of abuse portioning induces a high variability and low precision of analysis.

As described herein, incorporation of an affinity matrix that contains hydrophobic moieties on the surface thereof permits isolation of the target analyte found within the lipid structures thereby increasing sensitivity, yield, and precision. Such affinity matrices can be added into the collection vessel itself or can be added into a whole undiluted saliva sample received in the testing lab (onsite or added later). A non-limiting, exemplary affinity matrix comprises a non-imbibing, non-toxic affinity matrix composed of inert nylon thread to capture DOAs and metabolites contained within or on lipid or non-lipid bound structures. The affinity matrix is tailored to capture lipid bound DOAs by dyeing the matrix with hydrophobic and basic dyes that include but are not limited to: Sudan IV, Sudan Black B, Alizarin Cyanin, Crystal Violet, Safranin, Methylene Blue. The affinity matrix can be tailored to capture glycolic structures associated with DOAs and their metabolites by incorporating lectins or sugar (mannose) binding proteins on the surface of the affinity matrix.

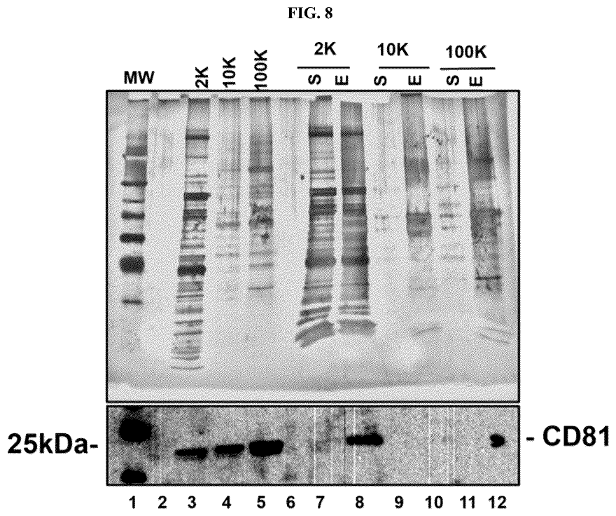

Isolation of extracellular vesicles (EVs), found in OF, has been demonstrated herein according to the methods described herein, using an affinity matrix comprising hydrophobic dye Sudan IV. The affinity matrix was mixed into different EV subtypes (2K, 10K, and 100K) and the depletion and elution of each EV by the affinity matrix was evaluated by silver stain analysis and by Western blot against the common EV tetraspanin marker CD81 (FIG. 8). The affinity matrix successfully depleted the EVs and concentrated them as evidence by CD81 expression.

### Enumerated Embodiments

The following exemplary embodiments are provided, the numbering of which is not to be construed as designating levels of importance:

Embodiment 1 provides a compound of formula (I), or a salt, solvate, stereoisomer, isotopologue, or metabolite thereof:

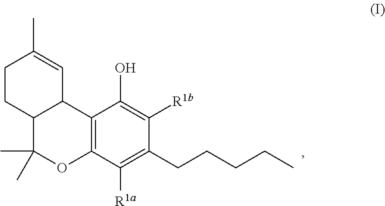

wherein:
R^{1a} and R^{1b} are each independently selected from the group consisting of H and

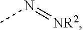

wherein at least one of R^{1a} and R^{1b} is

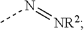

each occurrence of R² is independently selected from the group consisting of optionally substituted C₆-C₁₀ aryl and optionally substituted C₂-C₁₀ heteroaryl.

Embodiment 2 provides the compound of Embodiment 1, wherein the compound of formula (I) is selected from the group consisting of:

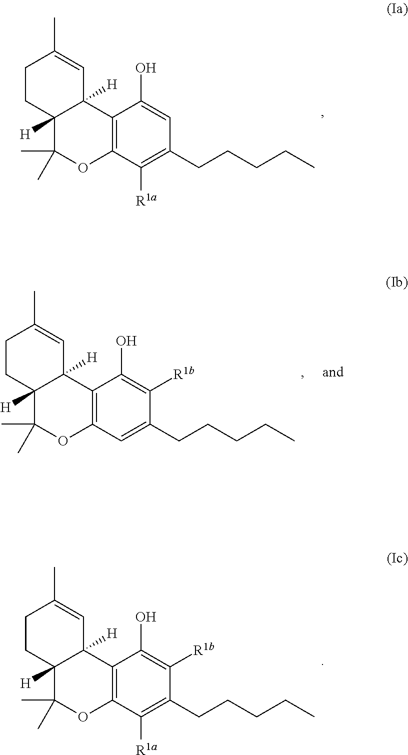

Embodiment 3 provides the compound of Embodiment 1 or 2, wherein each occurrence of R² is independently:

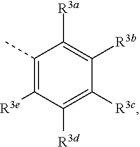

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, CI, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

Embodiment 4 provides the compound of Embodiment 3, wherein R^{3a}, R^{3b}, R^{3c}, R^{3d}, and R^{3e} are each independently selected from the group consisting of H, CH₃, OMe, F, Cl, Br, I, S(═O)₂OH, CN, and NO₂.

Embodiment 5 provides a composition comprising at least one drug of addiction (DOA), or a metabolite thereof, and at least one additive selected from the group consisting of an antifoaming agent, an acidic buffer, a surfactant or detergent, a mucolytic agent, an enzymatic reagent, and a mild antimicrobial agent.

Embodiment 6 provides the composition of Embodiment 5, wherein at least one of the following applies:
(a) the acidic buffer comprises at least one selected from the group consisting of acetic acid, ascorbic acid, and citric acid;
(b) the surfactant or detergent is a non-ionic surfactant selected from the group consisting of Triton X-100, Tween-20, sodium dodecyl sulfate (SDS), and NP-40;
(c) the surfactant or detergent is a polymeric surfactant or detergent selected from the group consisting of poloxamer 407 (P407), poloxamer (P188), poloxamer (P85), poloxamer 123 (P123), polyethylene glycol (PEG)-60, PEG-400, and PEG-1000;
(d) the mucolytic agent is selected from the group consisting of dithiothreitol and NaIO₄; and
(e) the enzymatic agent is an amylase.

Embodiment 7 provides the composition of Embodiment 6 wherein the at least one DOA, or a metabolite thereof, is in solution or associated with a lipid containing particle.

Embodiment 8 provides the composition of Embodiment 6 or 7, wherein at least one of the following applies:
(a) the surfactant or detergent comprises about 0.1% to about 5.0% of the composition;
(b) the acidic buffer comprises about 1.0% to about 5.0% of the composition;
(c) the mucolytic agent comprises about 1.0% to about 5.0% of the composition; and
(d) the antimicrobial agent comprises about 0.005% to about 0.150% of the composition.

Embodiment 9 provides the composition of any one of Embodiments 6-8, wherein the drug of addiction, or a metabolite thereof, is at least one selected from the group consisting of amphetamine, benzoylecgonine, benzodiazepines (e.g., alprazolam), cocaine, codeine, fentanyl, heroin (diacetylmorphine), hydrocodone, ketamine, methamphetamine, methadone, morphine, methylenedioxymethamphetamine (MDMA), oxycodone, phencyclidine, and tetrahydrocannabinol (THC).

Embodiment 10 provides a method for stabilizing and/or improving extractability of tetrahydrocannabinol (THC), or a metabolite thereof, in an oral fluid, the method comprising contacting an oral fluid comprising THC with at least one compound of formula (II):

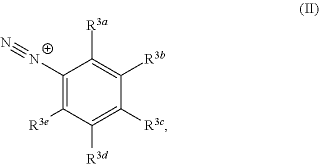

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R_{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

Embodiment 11 provides a method for detecting tetrahydrocannabinol (THC), or a metabolite thereof, in an oral fluid of a subject, the method comprising:
(a) contacting an oral fluid obtained from the subject with at least one compound of formula (II) to provide a stabilized sample:

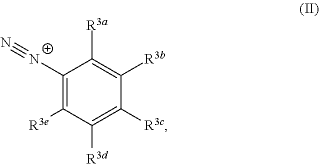

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O) (C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl;
(b) applying the stabilized sample to a lateral flow assay device comprising a band comprising an immobilized THC-binding antibody; and
(c) observing coloration of the band corresponding to the immobilized THC-binding antibody.

Embodiment 12 provides a method for detecting an analyte, or a metabolite thereof, in an oral fluid of a subject, the method comprising:
(a) combining an oral fluid obtained from the subject with at least one additive selected from the group consisting of an antifoaming agent, an acidic buffer, a surfactant or detergent, a mucolytic agent, an enzymatic reagent, and a mild antimicrobial agent to provide a stabilized sample;
(b) applying the stabilized sample to a lateral flow assay device comprising at least one band comprising an immobilized antibody which specifically binds to the analyte; and
(c) observing coloration of the band corresponding to the immobilized antibody which specifically binds to the analyte.

Embodiment 13 provides a method for at least partially sequestering an analyte from an oral fluid, the method comprising:
(a) contacting the oral fluid with a porous adsorbing agent, wherein the porous adsorbing agent comprises an inert, high surface area material covalently or noncovalently associated with an affinity label; and
(b) binding the analyte to the affinity label to form an analyte bound complex.

Embodiment 14 provides the method of Embodiment 13, further comprising:
(c) washing analyte bound complex with a suitable washing solvent; and
(d) eluting the analyte from the analyte bound complex with a suitable eluting solvent.

Embodiment 15 provides the method of Embodiment 14, wherein one of the following applies:
(a) the analyte is nonpolar, the suitable washing solvent is polar, and the suitable eluting solvent is nonpolar; or
(b) the analyte is polar or ionic, and the suitable washing solvent is nonpolar, and the suitable eluting solvent is polar.

Embodiment 16 provides the method of any one of Embodiments 13-15, wherein the porous adsorbing agent comprises an affinity matrix comprising a non-water imbibing, biocompatible filament, optionally wherein the non-water imbibing, biocompatible filament is a nylon filament.

Embodiment 17 provides the method of any one of Embodiments 13-15, wherein the porous adsorbing agent comprises a hydrogel, optionally wherein the hydrogel is a nanoparticle.

Embodiment 18 provides the method of any one of Embodiments 13-17, wherein the affinity ligand is selected from the group consisting of a dye and a lectin.

Embodiment 19 provides the method of Embodiment 18, wherein the dye is selected from the group consisting of an acidic dye, a basic dye, a fast dye, a metallic dye, a hydrophobic dye, and an uncharged polar dye.

Embodiment 20 provides the method of Embodiment 18 or 19, wherein the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

Embodiment 21 provides a method for quantifying an amount of analyte in a sample, the method comprising:
(a) adding to the sample a known amount of at least one isotopically labeled analog of the analyte to obtain a mixture;
(b) measuring relative intensity of the analyte and relative intensity of the isotopically labeled analog of the analyte by mass spectrometry;
(c) calculating an observed relative intensity ratio of the analyte to the isotopically labeled analog of the analyte (observed L:H ratio);
(d) comparing the observed L:H ratio to a standard curve L:H ratio.

Embodiment 22 provides the method of Embodiment 21, wherein the sample is processed after addition of the at least one isotopically labeled analog of the analyte.

Embodiment 23 provides the method of Embodiment 22, wherein the processing comprises at least one of the following:
(a) filtration of particulate matter from the sample;
(b) dilution of the sample with a stabilizing buffer;
(c) dilution of the sample with a pH buffer; and
(d) silica gel chromatography of the sample.

Embodiment 24 provides the method of Embodiment 23, wherein the stabilization buffer comprises a surfactant or detergent, optionally wherein the stabilization buffer comprises at least one selected from the group consisting of 3-((3-cholamidopropyl)dimethylammonio)-1-propanesulfonate (CHAPS), RapiGest, Tween-20, and NP-40.

Embodiment 25 provides the method of Embodiment 23 or 24, wherein the pH buffer comprises ammonium hydroxide or formic acid.

Embodiment 26 provides the method of any one of Embodiments 23-25, wherein the sample is loaded onto a silica gel column and eluted with a solvent, optionally wherein the solvent is selected from the group consisting of hexanes, ethyl acetate, methyl tert-butyl ether (MTBE), dichloromethane, and iso-propyl alcohol, or a mixture thereof.

Embodiment 27 provides the method of any one of Embodiments 23-26, wherein the eluted sample is dried under inert gas, optionally wherein the sample is dried at a temperature of about 40° C.

Embodiment 28 provides an oral fluid storage device comprising a porous adsorbing agent comprising an inert, high surface area material covalently or noncovalently associated with an affinity label.

Embodiment 29 provides the device of Embodiment 28, wherein the device is a sealable vessel.

Embodiment 30 provides the device of Embodiment 28 or 29, wherein the porous adsorbing agent is contained in the sealable vessel.

Embodiment 31 provides the device of any one of Embodiments 28-30, further comprising at least one compound of formula (II):

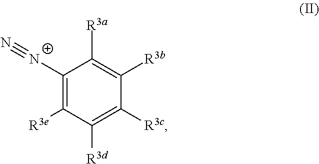

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O) (C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

Embodiment 32 provides the device of any one of Embodiments 28-31, wherein the device comprises a sealable inlet, optionally wherein the device further comprises a sealable outlet.

Embodiment 33 provides the device of Embodiment 32, wherein the device comprises an optionally removable filter positioned at the sealable inlet and/or at the sealable outlet.

Embodiment 34 provides an oral fluid storage device comprising at least one compound of formula (II):

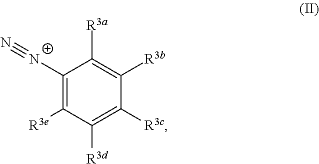

wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

Embodiment 35 provides the device of Embodiment 34, wherein the device is a sealable vessel.

Embodiment 36 provides an oral fluid storage device comprising at least one additive selected from the group consisting of an antifoaming agent, an acidic buffer, a surfactant or detergent, a mucolytic agent, an enzymatic reagent, and a mild antimicrobial agent.

Embodiment 37 provides the device of Embodiment 36, wherein the device is a sealable vessel.

Embodiment 38 provides a kit comprising the device of any one of Embodiments 28-33 and a cotton swab.

Embodiment 39 provides a kit comprising the device of Embodiment 34 or 35 and at least one lateral flow assay device for a drug of addiction (DOA), optionally wherein the kit further comprises a cotton swab.

Embodiment 40 provides a kit comprising the device of Embodiment 36 or 37 and at least one lateral flow assay device for a drug of addiction (DOA), optionally wherein the kit further comprises a cotton swab.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound of formula (I), or a salt, solvate, stereoisomer, isotopologue, or metabolite thereof: 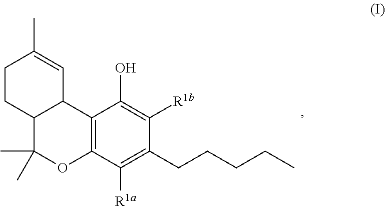
wherein:
R^{1a} and R^{1b} are each independently selected from the group consisting of H and 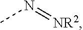
wherein at least one of R^{1a} and R^{1b} is 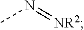
each occurrence of R² is independently selected from the group consisting of optionally substituted C₆-C₁₀ aryl and optionally substituted C₂-C₁₀ heteroaryl.

2. The compound of claim 1, wherein the compound of formula (I) is selected from the group consisting of:

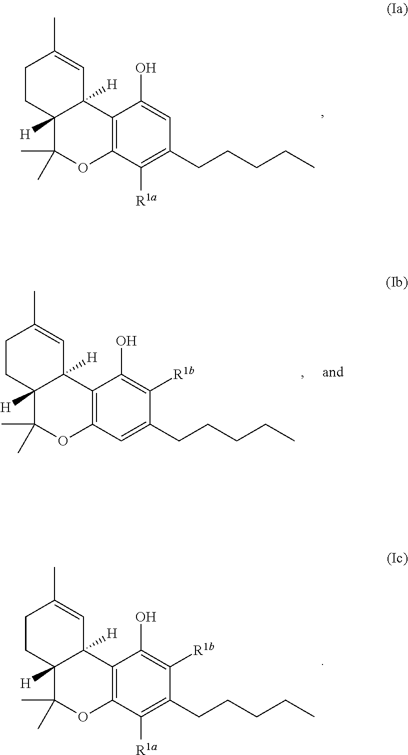

3. The compound of claim 1, wherein each occurrence of R² is independently: 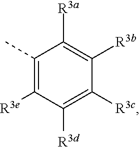
wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

4. The compound of claim 3, wherein R^{3a}, R^{3b}, R^{3c}, R^{3d}, and R^{3e} are each independently selected from the group consisting of H, CH₃, OMe, F, Cl, Br, I, S(═O)₂OH, CN, and NO₂.

5. A composition comprising at least one drug of addiction (DOA), or a metabolite thereof, and at least one additive selected from the group consisting of a silicon based antifoaming agent, an acidic buffer, a surfactant or detergent, a mucolytic agent, an enzymatic reagent, and a mild antimicrobial agent.

6. The composition of claim 5, wherein at least one of the following applies:
(a) the acidic buffer comprises at least one selected from the group consisting of acetic acid, ascorbic acid, and citric acid;
(b) the surfactant or detergent is a non-ionic surfactant selected from the group consisting of Triton X-100, Tween-20, sodium dodecyl sulfate (SDS), and NP-40;
(c) the surfactant or detergent is a polymeric surfactant or detergent selected from the group consisting of poloxamer 407 (P407), poloxamer (P188), poloxamer (P85), poloxamer 123 (P123), polyethylene glycol (PEG)-60, PEG-400, and PEG-1000;
(d) the mucolytic agent is selected from the group consisting of dithiothreitol and NaIO₄; and
(e) the enzymatic agent is an amylase.

7. The composition of claim 6, wherein the at least one DOA, or a metabolite thereof, is in solution or associated with a lipid containing particle.

8. The composition of claim 6, wherein at least one of the following applies:
(a) the surfactant or detergent comprises about 0.1% to about 5.0% of the composition;
(b) the acidic buffer comprises about 1.0% to about 5.0% of the composition;
(c) the mucolytic agent comprises about 1.0% to about 5.0% of the composition; and
(d) the antimicrobial agent comprises about 0.005% to about 0.150% of the composition.

9. The composition of claim 6, wherein the drug of addiction, or a metabolite thereof, is at least one selected from the group consisting of amphetamine, benzoylecgonine, benzodiazepines (e.g., alprazolam), cocaine, codeine, fentanyl, heroin (diacetylmorphine), hydrocodone, ketamine, methamphetamine, methadone, morphine, methylenedioxymethamphetamine (MDMA), oxycodone, phencyclidine, and tetrahydrocannabinol (THC).

10. A method for stabilizing and/or improving extractability of tetrahydrocannabinol (THC), or a metabolite thereof, in an oral fluid, the method comprising contacting an oral fluid comprising THC with at least one compound of formula (II): 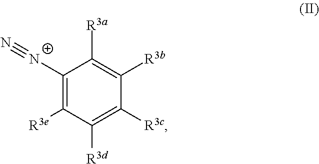
wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

11. A method for detecting tetrahydrocannabinol (THC), or a metabolite thereof, in an oral fluid of a subject, the method comprising:
(a) contacting an oral fluid obtained from the subject with at least one compound of formula (II) to provide a stabilized sample: 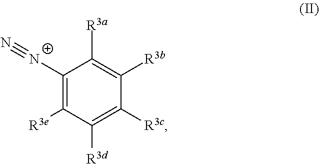
wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl;
(b) applying the stabilized sample to a lateral flow assay device comprising a band comprising an immobilized THC-binding antibody; and
(c) observing coloration of the band corresponding to the immobilized THC-binding antibody.

12. A method for detecting an analyte, or a metabolite thereof, in an oral fluid of a subject, the method comprising:
(a) combining an oral fluid obtained from the subject with at least one additive selected from the group consisting of an antifoaming agent, an acidic buffer, a surfactant or detergent, a mucolytic agent, an enzymatic reagent, and a mild antimicrobial agent to provide a stabilized sample;
(b) applying the stabilized sample to a lateral flow assay device comprising at least one band comprising an immobilized antibody which specifically binds to the analyte; and
(c) observing coloration of the band corresponding to the immobilized antibody which specifically binds to the analyte.

13. A method for at least partially sequestering an analyte from an oral fluid, the method comprising:
(a) contacting the oral fluid with a porous adsorbing agent,
wherein the porous adsorbing agent comprises an inert, high surface area material covalently or noncovalently associated with an affinity label; and
(b) binding the analyte to the affinity label to form an analyte bound complex.

14. The method of claim 13, further comprising:
(c) washing analyte bound complex with a suitable washing solvent; and
(d) eluting the analyte from the analyte bound complex with a suitable eluting solvent.

15. The method of claim 14, wherein one of the following applies:
(a) the analyte is nonpolar, the suitable washing solvent is polar, and the suitable eluting solvent is nonpolar; or
(b) the analyte is polar or ionic, and the suitable washing solvent is nonpolar, and the suitable eluting solvent is polar.

16. The method of claim 13, wherein the porous adsorbing agent comprises an affinity matrix comprising a non-water imbibing, biocompatible filament, optionally wherein the non-water imbibing, biocompatible filament is a nylon filament.

17. The method of claim 13, wherein the porous adsorbing agent comprises a hydrogel, optionally wherein the hydrogel is a nanoparticle.

18. The method of claim 13, wherein the affinity ligand is selected from the group consisting of a dye and a lectin.

19. The method of claim 18, wherein the dye is selected from the group consisting of an acidic dye, a basic dye, a fast dye, a metallic dye, a hydrophobic dye, and an uncharged polar dye.

20. The method of claim 18, wherein the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

21. A method for quantifying an amount of analyte in a sample, the method comprising:
(a) adding to the sample a known amount of at least one isotopically labeled analog of the analyte to obtain a mixture;
(b) measuring relative intensity of the analyte and relative intensity of the isotopically labeled analog of the analyte by mass spectrometry;
(c) calculating an observed relative intensity ratio of the analyte to the isotopically labeled analog of the analyte (observed L:H ratio);
(d) comparing the observed L:H ratio to a standard curve L:H ratio.

22. The method of claim 21, wherein the sample is processed after addition of the at least one isotopically labeled analog of the analyte.

23. The method of claim 22, wherein the processing comprises at least one of the following:
(a) filtration of particulate matter from the sample;
(b) dilution of the sample with a stabilizing buffer;
(c) dilution of the sample with a pH buffer; and
(d) silica gel chromatography of the sample.

24. The method of claim 23, wherein the stabilization buffer comprises a surfactant or detergent, optionally wherein the stabilization buffer comprises at least one selected from the group consisting of 3-((3-cholamidopropyl) dimethylammonio)-1-propanesulfonate (CHAPS), RapiGest, Tween-20, and NP-40.

25. The method of claim 23, wherein the pH buffer comprises ammonium hydroxide or formic acid.

26. The method of claim 23, wherein the sample is loaded onto a silica gel column and eluted with a solvent, optionally wherein the solvent is selected from the group consisting of hexanes, ethyl acetate, methyl tert-butyl ether (MTBE), dichloromethane, and iso-propyl alcohol, or a mixture thereof.

27. The method of claim 23, wherein the eluted sample is dried under inert gas, optionally wherein the sample is dried at a temperature of about 40° C.

28. An oral fluid storage device comprising a porous adsorbing agent comprising an inert, high surface area material covalently or noncovalently associated with an affinity label.

29. The device of claim 28, wherein the device is a sealable vessel.

30. The device of claim 29, wherein the porous adsorbing agent is contained in the sealable vessel.

31. The device of claim 28, further comprising at least one compound of formula (II): 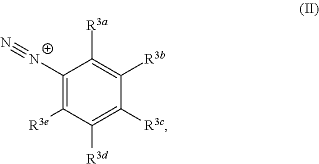
wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O) (C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

32. The device of claim 28, wherein the device comprises a sealable inlet, optionally wherein the device further comprises a sealable outlet.

33. The device of claim 32, wherein the device comprises an optionally removable filter positioned at the sealable inlet and/or at the sealable outlet.

34. An oral fluid storage device comprising at least one compound of formula (II): 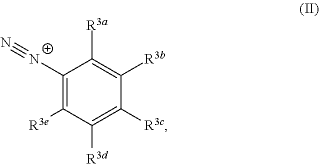
wherein:
each occurrence of R^{3a}, R^{3b}, R^{3c}, R^{3d}, and R^{3e} is independently selected from the group consisting of H, C₁-C₆ alkyl, C₃-C₈ cycloalkyl, C₂-C₆ alkenyl, C₂-C₆ alkynyl, C₆-C₁₀ aryl, C₂-C₁₀ heteroaryl, Fl, Cl, Br, I, CN, NO₂, OR^{A}, N(R^{A})(R^{B}), C(═O)R^{A}, C(═O)OR^{A}, C(═O)N(R^{A})(R^{B}), OC(═O)R^{A}, N(R^{A})C(═O)R^{B}, S(═O)R^{A}, S(═O)₂OR^{A}, S(═O)₂N(R^{A})(R^{B}), and OP(═O)(OR^{A})(OR^{B});
each occurrence of R^{A} and R^{B} is independently selected from the group consisting of H, —C(═O)(C₁-C₆ alkyl), C₁-C₆ alkyl, C₁-C₆ haloalkyl, C₃-C₈ cycloalkyl, C₇-C₁₂ aralkyl, phenyl, naphthyl, and heteroaryl.

35. The device of claim 34, wherein the device is a sealable vessel.

36. An oral fluid storage device comprising at least one additive selected from the group consisting of an antifoaming agent, an acidic buffer, a surfactant or detergent, a mucolytic agent, an enzymatic reagent, and a mild antimicrobial agent.

37. The device of claim 36, wherein the device is a sealable vessel.

38. A kit comprising the device of claim 28 and a cotton swab.

39. A kit comprising the device of claim 34 and at least one lateral flow assay device for a drug of addiction (DOA), optionally wherein the kit further comprises a cotton swab.

40. A kit comprising the device of claim 36 and at least one lateral flow assay device for a drug of addiction (DOA), optionally wherein the kit further comprises a cotton swab.
